(19) United States
(12) Patent Application Publication
(10) Pub. No.: US 2026/0250585 A1
(43) Pub. Date: Aug. 27, 2026

(54) SYSTEMS AND PROCESS FOR THE PRODUCTION OF HYDROCARBON PRODUCTS

(71) Applicant: SABIC GLOBAL TECHNOLOGIES B.V., BERGEN OP ZOOM (NL)

(72) Inventor: Kenneth Francis LAWSON, Bangalore (IN)

(73) Assignee: SABIC GLOBAL TECHNOLOGIES B.V., BERGEN OP ZOOM (NL)

(21) Appl. No.: 18/992,565

(22) PCT Filed: Jul. 6, 2023
(86) PCT No.: PCT/EP2023/068778

§ 371 Date: Jan. 8, 2025

(30) Foreign Application Priority Data
Jul. 9, 2022 (IN) 202241039487

(51) Int. Cl. C10G 69/06; C07C 43/04; C10G 69/08

(52) U.S. Cl. CPC C10G 69/06; C07C 43/046; C10G 69/08; C10G 2300/1033; C10G 2400/20

(57) ABSTRACT

Systems and methods for the production of petroleum products from crude oil are disclosed. A system can include a crude oil processing unit, a steam cracking, and a gaseous hydrocarbon separation unit coupled to the crude oil processing unit and steam cracking unit. Naphtha and/or light hydrocarbons can be separated in the gaseous hydrocarbon separation unit and further processed in the steam cracking unit or stored for future processing.

### CROSS REFERENCE TO RELATED APPLICATIONS

None.

### BACKGROUND OF THE INVENTION

### A. Field of the Invention

The invention generally concerns production of hydrocarbon products from a hydrocarbon feedstock. A system can include a crude oil processing unit, a gaseous hydrocarbon unit, and a steam cracking unit. The gaseous hydrocarbon unit can process hydrocarbon having a boiling point less than 200° C. received from the crude oil processing unit and produce naphtha, C4 hydrocarbons, and a C2-C3 hydrocarbon stream. The C2-C3 hydrocarbon stream can be cracked in the steam cracking unit to produce ethylene and propylene. The C4 hydrocarbons and naphtha can be stored for further use or sale.

### B. Description of Related Art

Olefins (e.g., ethylene), are basic building blocks for a variety of commercially valuable polymers. Since naturally occurring sources of olefins do not exist in commercial quantities, polymer producers rely on methods for converting the more abundant lower alkanes into olefins. The method of choice for today's commercial scale producers is steam cracking, a highly endothermic process where steam-diluted alkanes are subjected briefly to a temperature of at least 800° C. The fuel demand to produce the required temperatures and the need for equipment that can withstand that temperature add significantly to the overall cost. Also, the high temperature promotes the formation of coke which accumulates within the system, resulting in the need for costly periodic reactor shut-down for maintenance and coke removal.

Processes and systems to produce olefins from crude oil have been described. For example, International Patent Application No. WO 2017/133975 to Ward et al., describes an integrated process to convert crude oil into petrochemical products that includes crude oil distillation, hydrocracking, and steam cracking to produce petroleum products. Some processes use a Mixed Feed Cracker (MFC). A MFC can handle from light hydrocarbons such as ethane, propane, and butane through naphtha up to heavy liquid feedstocks such as gas oils and hydrocracker residues to produce gasoline and a majority of petrochemical industry products. A MFC process can be operated depending on the optimized downstream value chain between the oil or gas availability as feedstock and the market prices in high added value hydrocarbon products. However, mixed feed steam crackers suffer in that they do no convert all material to olefins. Optimal performance of cracking units occurs when fed with compatible feeds. In most steam cracking units, a dedicated furnace is usually required for light hydrocarbons. This results in different types and sizes of furnaces.

Also, steam cracking can result in the slow deposition of coke, a form of carbon, on the reactor walls. Decoking requires the furnace to be isolated from the process and then a flow of steam or a steam/air mixture is passed through the furnace coils. This converts the hard solid carbon layer to carbon monoxide and carbon dioxide. Once this reaction is complete, the furnace is returned to service. Due to the amount of coking that occurs in the MRC when hydrocarbons are cracked, the MRC generally requires at least two compatible furnaces for the cracking of specific hydrocarbons, which can compromise the size of the furnaces in the MRC and make the process less efficient and more cost intensive.

Systems and processes to produce hydrocarbon products have been described. For example, U.S. Pat. No. 11,180,706 to Al-Sayed et al. describes a configuration for olefins production. The processes progressively separate a crude oil into light and heavy fractions, which can be upgraded using fixed bed hydroconversion unit, a fluidized catalytic conversion unit, or a residue hydrocracking unit. The upgraded fluids can be fed to a steam cracking unit for production of olefins. This process suffers from less than optimal performance, which can increase capital cost of the overall process and lower profitability.

Overall, while the technologies of producing olefins, namely ethylene, exist, they can be energy inefficient and expensive.

### SUMMARY OF THE INVENTION

A discovery has been made that provides a solution to at least one of the problems associated with production of hydrocarbon products. In one aspect, the discovery can include a system that includes a hydrocarbon processing unit that is capable of receiving crude oil feed, a gas separation unit, and a steam cracking unit. The hydrocarbon processing unit can include a crude distillation unit and/or a vacuum distillation unit. This configuration allows for the advantages of 1) lower cracking temperatures in the steam cracking unit and 2) increased ethylene selectivity. Without wishing to be bound by theory, it is believed that limiting the amount of naphtha and C4 hydrocarbons in the steam cracking unit lowers the vapor pressure in the unit thus allowing C4+ hydrocarbons to be converted to ethylene at higher conversion and selectivity.

In one aspect of the present invention, systems and processes to produce hydrocarbon products are described. One system can include a crude oil processing unit, a gaseous hydrocarbon separation unit, a steam cracking unit, and at least one storage unit. The crude oil processing unit can be capable of producing a gaseous hydrocarbon stream that includes hydrocarbons having a boiling point less than 200° C., preferably less than 180° C., a gas oil stream, and a heavy hydrocarbon stream. The crude oil processing unit can include at least one crude oil distillation unit (CDU) and/or at least one vacuum distillation unit (VDU). The gaseous hydrocarbon separation unit can be capable of receiving the gaseous hydrocarbon stream that includes hydrocarbons having a boiling point less than 200° C. produced from the crude oil processing unit and can be capable of producing a naphtha stream, a gaseous stream that can include C4 hydrocarbons, and a C2-C3 hydrocarbon stream. The steam cracking unit can be capable of receiving the C2-C3 hydrocarbon unit and cracking the C2-C3 hydrocarbon stream to produce ethylene and propylene. In one aspect, a butane storage unit can be provided for storing the C4 hydrocarbons produced from the gaseous hydrocarbon separation unit and/or a naphtha storage unit can be provided for storing the naphtha produced from the gaseous hydrocarbon separation unit. The system can also include a butene hydrogenation unit positioned downstream from and indirectly coupled to the steam cracking unit. The butene hydrogenation unit can be capable of producing butane from butenes. In some aspects, the system can include a resid hydrocracking unit capable of receiving the heavy stream and configured to produce naphtha from the heavy hydrocarbon stream. The produced naphtha can be provided the steam cracking unit. The steam cracking unit can be capable of cracking the naphtha at a different temperature than the cracking temperature for the C2-C3 hydrocarbons. In some aspects, the system includes a hydrocracking unit capable of receiving the gas oil and configured to producing naphtha and unconverted oil (UCO) from the gas oil. UCO is different from hydrocracking feed as it has been converted by hydro-treatment and includes saturated hydrocarbons. The produced naphtha can be provided to the steam cracking unit via a second naphtha conduit.

Processes to produce hydrocarbon products using the systems of the present invention are also described. In one aspect, a process for the production of hydrocarbons can include (a) subjecting a crude oil hydrocarbon feed to conditions sufficient to produce a gaseous hydrocarbon stream that can include hydrocarbons have a boiling point less than 200° C., preferably 180° C., a gas oil stream, and a heavy hydrocarbon stream. Conditions in step (a) can include a temperature of 50° C. to 700° C. In step (b), the gaseous hydrocarbon stream can be separated into a C2-C3 hydrocarbon stream, a C4 hydrocarbon stream, and a naphtha stream in the gaseous hydrocarbon separation unit. The gaseous hydrocarbon stream of has a sulfur content of less than 500 ppm, preferably less than 300 ppm. The C4 hydrocarbons can be stored in a storage unit. Naphtha can also be stored in a storage unit. In step (c), the C2-C3 hydrocarbon can be subjected to steam cracking conditions sufficient to produce an ethylene product stream and a propylene product stream. In one aspect, the process can include subjecting the gas oil to conditions sufficient to produce a second naphtha composition and UCO. The second naphtha composition, UCO, or a blend thereof can be provided to the steam cracking unit of step (c) to continue the process. In some aspects, the heavy hydrocarbons in step (a) can be subjected to conditions sufficient to produce a third naphtha composition and pitch. The third naphtha composition can be provided to the steam cracking unit of step (c) to further the process of producing hydrocarbon products. In the steam cracking unit, the second and third naphtha compositions, UCO, or a blend thereof, can be subjected to conditions sufficient to produce second C4 hydrocarbon composition, ethylene, and propylene, or a combination thereof. Such conditions can be the same or different than the cracking conditions for the C2-C3 hydrocarbons. The second C4 hydrocarbon composition can be processed to produce MTBE. The process can also include providing one or more C4 hydrocarbons compositions produced from a butene hydrogenation unit to the steam cracking unit of step (c). The second and third naphtha compositions, UCO, or a combination thereof can be subjected to conditions in the steam cracking unit sufficient to produce a second C4 hydrocarbon composition, ethylene, and propylene, or a combination thereof.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to other aspects of the invention. It is contemplated that any embodiment or aspect discussed herein can be combined with other embodiments or aspects discussed herein and/or implemented with respect to any method or composition of the invention, and vice versa. Furthermore, compositions of the invention can be used to achieve methods of the invention.

The following includes definitions of various terms and phrases used throughout this specification.

The term “C# hydrocarbons”, wherein “#” is a positive integer, is meant to describe all hydrocarbons having #carbon atoms. Moreover, the term “C#+ hydrocarbons” is meant to describe all hydrocarbon molecules having #or more carbon atoms. Accordingly, the term “C2+ hydrocarbons” is meant to describe a mixture of hydrocarbons having 2 or more carbon atoms. The term “C2+ alkanes” accordingly relates to alkanes having 2 or more carbon atoms.

The terms “about” or “approximately” are defined as being close to as understood by one of ordinary skill in the art. In one non-limiting embodiment, the terms are defined to be within 10%, preferably within 5%, more preferably within 1%, and most preferably within 0.5%.

The terms “wt. %”, “vol. %”, or “mol. %” refers to a weight percentage of a component, a volume percentage of a component, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The systems and processes of the present invention can “comprise,” “consist essentially of,” or “consist of” particular ingredients, components, compositions, etc. disclosed throughout the specification. With respect to the transitional phrase “consisting essentially of,” in one non-limiting aspect, a basic and novel characteristic of the systems and processes of the present invention are their abilities to produce a variety of petrochemical products.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

Advantages of the present invention may become apparent to those skilled in the art with the benefit of the following detailed description and upon reference to the accompanying drawings.

FIG. 1 illustrates an embodiment of a system to produce hydrocarbon products from a crude feed that includes a crude oil processing unit coupled to a gaseous hydrocarbon separation unit, and a steam cracking unit coupled to the crude oil processing unit and the gaseous hydrocarbon separation unit.

FIG. 2 is an illustration of the system of FIG. 1 with additional processing units shown.

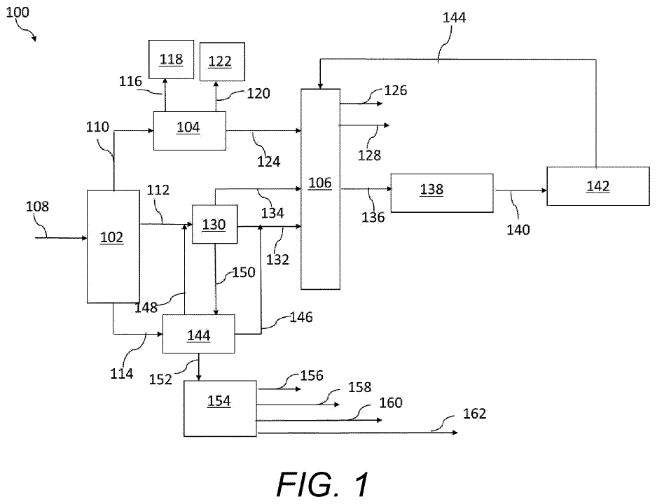

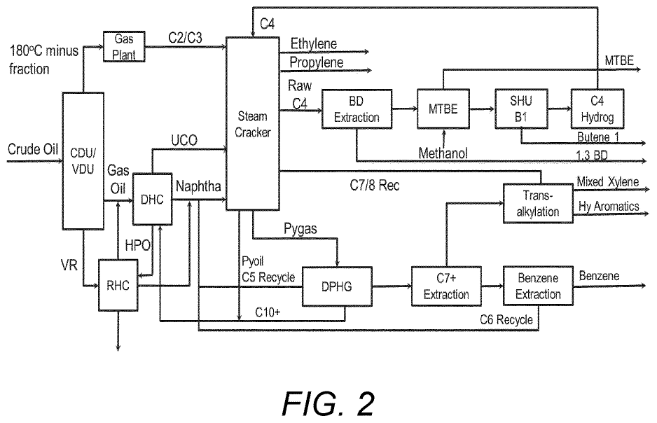

While the invention is susceptible to various modifications and alternative forms, specific embodiments thereof are shown by way of example in the drawings. The drawings may not be to scale. Still further, the schematics illustrated in FIGS. 1 and 2 can be combined with one another, which can be used, for example, to create a more robust process of producing a variety of petrochemical products from crude oil.

### DETAILED DESCRIPTION OF THE INVENTION

A discovery has been made that provides a solution to at least one of the problems associated with producing hydrocarbon products from crude oil. In one aspect, the crude oil can be processed in a crude oil processing unit to produce a hydrocarbon stream having a boiling temperature of less than 200° C., preferably less than 180° C., gas oil and heavy hydrocarbons. The gas oil can be provided to the steam cracking unit for further processing. By limiting the amount of naphtha and C4 hydrocarbons fed to the steam cracking unit, the vapor pressure control in the steam cracking unit can be controlled and heavier streams (e.g., gas oil) higher selectivity to ethylene can be achieved. Thus, providing a more cost and/or energy efficient system. These and other non-limiting aspects of the present invention are discussed in further detail in the following sections with reference to the Figures.

FIGS. 1 and 2 illustrate systems that produce petroleum products from crude oil. Referring to FIG. 1, system 100 for producing petroleum products is described. FIG. 2 illustrates system 100 and the processing of the methyl tert-butyl ether (MTBE) streams and other streams produced from the steam cracking unit. System 100 can include a crude oil processing unit 102, a gaseous hydrocarbon separation unit 104, and a steam cracking unit 106. Crude oil 108 enters feed separation 102. Crude oil can be the petroleum extracted from geologic formations in its unrefined form. The term crude oil can also include petroleum that has been subjected to water-oil separations and/or gas-oil separation and/or desalting and/or stabilization. Non-limiting examples of crude oil include Arabian Heavy, Arabian Light, other Gulf crudes, Brent, North Sea crudes, North and West African crudes, Indonesian, Chinese crudes, West Texas crude, and mixtures thereof, but also shale oil, tar sands, gas condensates and bio-based oils. The crude oil used as feed to the process of the present invention preferably is conventional petroleum having an API gravity of more than 20° API as measured by the ASTM D287 standard. In one aspect, the crude oil used in the process of the present invention is a light crude oil having an API gravity of more than 30° API. In another aspect, the crude oil used in the process of the present invention can include Arabian Light Crude Oil. Arabian Light Crude Oil typically has an API gravity of between 32-36° API and a sulfur content of between 1.5-4.5 wt. %.

In crude oil processing unit 102, crude oil 108 is processed into a light hydrocarbon stream 110, gas oil hydrocarbon stream 112, and vacuum resid (heavy hydrocarbons) stream 114. Light hydrocarbon stream can have a boiling temperature of less than 200° C., preferably less than 180° C. (e.g., 200° C., 195° C., 190° C., 180° C., 175° C. or less, or any range or value there between). Gas oil can be have a boiling point range of about 340-560° C., more preferably of about 350-550° C., more preferably 250-360° C., most preferably of about 260-350° C. Vacuum resid (heavy hydrocarbons) can have a boiling point of more than about 340° C., more preferably of more than about 350° C. Crude oil processing unit 102 can include a flash vessel, one or more fractionating columns capable of process crude oil 108 into one or more fractions based on boiling point range. In some embodiments, crude oil processing unit 102 can include a vacuum distillation unit to further separate the resid into a vacuum gas oil fraction and vacuum residue fraction. In case vacuum distillation is used, the vacuum gas oil fraction and vacuum residue (heavy hydrocarbons) fraction may be further processed in crude oil processing unit 102. For instance, the vacuum residue fraction may be specifically subjected to solvent deasphalting before further processing. In other embodiments, slurry resid hydrocracking may be used to process crude oil 108 into fractions.

Light hydrocarbon stream 110 can exit crude oil processing unit 102 and enter gaseous hydrocarbon separation unit 104. In gaseous hydrocarbon unit 104, light hydrocarbon stream 110 can be separated into naphtha, C4 hydrocarbons, and C2-C3 hydrocarbons. Gaseous hydrocarbon unit 104 can include one or more distillation units known in the art capable of separating hydrocarbons having a boiling temperature of less than 200° C. For example, a naphtha stream at a boiling range of 20° C. to 190° C., a C4 hydrocarbon stream at −10° C. to 0° C., preferably −5° C. a C2/C3 hydrocarbon stream at −90 to −40° C. In some embodiments, gaseous hydrocarbon separation unit can include one or more units capable of removing sulfur containing compounds from light hydrocarbon stream 110. Any conventional method suitable for the separation of the gases may be employed in the context of the present invention. Accordingly, the gases may be subjected to multiple compression stages wherein acid gases such as CO₂ and H₂S may be removed between compression stages. The sulfur level can be reduced to less than 500 ppm, preferably less than 300 ppm (e.g., 500, 450, 425, 400, 375, 350, 325, 300, 275, 250, 225, 200, 175, 150, 125, 100 ppm or less, or any range or value there between) using conventional methods. The light hydrocarbon can be processed (e.g., multiple distillations) to remove hydrogen and/or other impurities (e.g., CO2/H2S) and separated into naphtha, C4 hydrocarbons, and C2-C3 gases. Naphtha stream 116 can exit gaseous hydrocarbon separation unit 104 and enter storage unit 118. C4 hydrocarbon stream 120 can exit gaseous hydrocarbon separation unit 104 and enter storage unit 122.

C2-C3 hydrocarbon stream 124 can exit gaseous hydrocarbon separation unit 104 and enter steam cracking unit 106. In steam cracking unit 106, the C2-C3 feed can be subjected to steam cracking at a temperature of 600° C. to 900° C. (e.g., 600° C., 625° C., 650° C., 675° C., 700° C., 725° C., 750° C., 775° C., 850° C., 875° C., 900° C., or any value or range there between) and/or a pressure of 0.2 MPa to 0.3 MPa (e.g., 0.2 MPa, 0.21 MPa, 0.22 MPa, 0.23 MPa, 0.24 MPa, 0.25 MPa, 0.26 MPa, 0.27 MPa, 0.28 MPa, 0.30 MPa, or any value or range there between). At such a temperature and pressure the ethane C2-C3 hydrocarbons are cracked to make ethylene and propylene. In a steam cracking process, the saturated hydrocarbons are broken down into smaller, often unsaturated, hydrocarbons such as ethylene and propylene by diluting the mixed hydrocarbon feed with steam and heating the mixture in a furnace in the absence presence of oxygen. The steam cracking reaction can have a residence times of 50-1000 milliseconds. Steam cracking unit can have a fractionation unit (not shown) or a gas fractionation unit (not shown) capable of separating ethane and/or propane from the olefin product stream. Such fractionation units are well known in the art. Ethylene stream 126 can exit steam cracking unit 106, and be stored, sold, or used in other processing units. Propylene stream 128 can exit steam cracking unit 106, and be stored, sold, or used in other processing units.

Gas oil stream 112 can exit crude oil processing unit 102 and enter gas oil processing unit 130. In gas oil processing unit 130, gas oil stream 112 can be subjected to a various distillations processes, hydrocracking process, or a combination thereof to produce additional naphtha and a UCO.

Gas oil processing can include one or more fixed bed catalytic reactors with one or more fractionation units to separate desired products from unconverted material and can also incorporate the ability to recycle unconverted material to one or both of the reactors. Gas oil processing reactors may be operated at a temperature of 200-600° C., preferably 300-400° C., a pressure of 3-35 MPa, preferably 5 to 20 MPa together with 5-20 wt. % of hydrogen (in relation to the hydrocarbon feedstock). Catalysts used in such processes comprise one or more elements selected from the group consisting of Pd, Rh, Ru, Ir, Os, Cu, Co, Ni, Pt, Fe, Zn, Ga, In, Mo, W and V in metallic or metal sulfide form supported on an acidic solid such as alumina, silica, alumina-silica and zeolites. Naphtha stream 132 (second naphtha stream) and UCO stream 134 (UCO stream in FIG. 2) can each exit gas oil processing (DHC in FIG. 2) unit 130 and enter steam cracking unit 106 to continue the process to produce petroleum products (e.g., ethylene, propylene, and butane).

In steam cracking unit 106, naphtha stream 132 and UCO (UCO in FIG. 2) stream 134 are subjected to steam cracking conditions previously described to produce ethylene, propylene, and C4 hydrocarbons, Pygas and C7/8 hydrocarbons. The steam cracking unit can include one or more furnaces such that the naphtha and the UCO can be cracked at a separate temperature than the C2-C3 hydrocarbons. In some embodiments, the same furnace can be used. Pygas can include aromatics, olefins, and paraffins ranging from C5s to C12s. The C4 hydrocarbons can be a mixture of butadiene, butane, and butenes (e.g., ethyl acetylene, vinyl acetylene, 1,3-butadiene, 1,2-butadiene, isobutylene, cis-2-butene, trans-2-butene, 1-butene, isobutane, and n-butane). C4 hydrocarbons stream 136 can exit steam cracking unit 106 and enter MTBE processing system 138. In MTBE processing system, MTBE is produced in addition to an MTBE effluent. MTBE processing system can include a butadiene (BD) separation unit, a MTBE synthesis unit, a selective hydrogenation unit (SHU in FIG. 2) capable of hydrogenating a portion of the effluent from the MTBE process to produce 1-butene. MTBE effluent 140 can be further processed by passing it through C4 hydrogenation unit 142 to produce C4 alkanes (second C4 hydrocarbon stream). C4 hydrogenation unit can be any C4 hydrogenation unit known in the art. A portion or all of C4 alkane stream 144 can exit C4 hydrogenation unit and enter steam cracking unit 106 to further the production of ethylene and/or propylene. In some embodiments, a portion or all of C4 hydrocarbon stream 144 is provided to C4 hydrocarbon storage unit 118.

Vacuum resid stream 114 can exit crude oil processing unit 102 and enter resid hydrocracking unit 144. Resid hydrocracking unit 144 is capable of converting resid into naphtha and gas oil. Naphtha stream 146 (third naphtha stream) can exit reside hydrocracking unit 144 and be combined with naphtha stream 132 (second naphtha stream) or enter steam cracking unit 106 (not shown). Gas oil stream 148 can exit reside hydrocracking unit 144 and be combined with gas oil stream 112, or enter gas oil processing unit 130 to be further processed into hydrocarbon products (e.g., ethylene, propylene, C4 hydrocarbons, or a combination thereof). In gas oil processing unit 130, the gas oil and some lighter components (components ranging from C2 to long chain alkanes up to C24) from the reside hydrocracking unit 144 can be fed to a fixed bed hydrocracker unit for conversion to material suitable for feed to a steam cracker. The components made in the fixed bed hydrocracker can be separated in the hydrocracker unit or provided to gaseous separation unit 104 (not shown) where they can be combined with the light steam from the crude separation to segregate hydrocarbons having less than 4 carbon atoms (<C4), C4 hydrocarbons and heavier than C4 (>C4 hydrocarbons) from the mixture. More optimal steam cracker performance can be observed if the <C4 hydrocarbons are cracked at a different temperature than the >C4 hydrocarbons. Purge gas from the hydrocrackers may be routed to the steam cracker for recovery of useful components. Resid hydrocracking processes are well established. For example, three basic reactor types can be employed in commercial hydrocracking which are a fixed bed (trickle bed) reactor type, an ebullated bed reactor type and slurry (entrained flow) reactor type. Fixed bed resid hydrocracking processes are well-established and are capable of processing contaminated streams such as atmospheric residues and vacuum residues to produce the gas oil and naphtha. The catalysts used in fixed bed resid hydrocracking processes can include cobalt (CO), molybdenum (Mo), nickel (Ni), or a combination thereof on a refractory support, typically alumina. In case of highly contaminated feeds, the catalyst in fixed bed resid hydrocracking processes can also be replenished to a certain extend (moving bed). The process conditions can include a temperature of 350° C. to 450° C. and a pressure of 2 to 20 MPa. Ebullated bed resid hydrocracking processes are also well-established and are inter alia characterized in that the catalyst is continuously replaced allowing the processing of highly contaminated feeds. The catalysts used in ebullated bed resid hydrocracking processes can include Co, Mo, Ni, or a combination thereof on a refractory support, typically alumina. The process conditions can include a temperature of 350° C. to 450° C. and a pressure of 5 MPa to 25 MPa. Slurry resid hydrocracking processes represent a combination of thermal cracking and catalytic hydrogenation to achieve high yields of distillable products from heavy resid feeds that are often highly contaminated. Such slurry resid hydrocracking processes are known (for example, U.S. Pat. No. 5,932,090, US 2012/0234726 A1 and WO 2014142874 A1). In the first liquid stage, thermal cracking and hydrocracking reactions can occur simultaneously in the bubble slurry phase at process conditions that include a temperature of 400-500° C. and a pressure of 15-25 MPa gauge. Resid, hydrogen and catalyst are introduced at the bottom of the reactor and a bubble slurry phase can be formed; the height of which depends on flow rate and desired conversion. In these processes, catalyst can be continuously replaced to achieve consistent conversion levels through an operating cycle. The catalyst can be an unsupported metal sulfide that is generated in situ within the reactor. The heavy-distillate produced by resid upgrading can be recycled to the resid hydrocracking unit 144 until extinction. In addition, higher boiling hydrocarbons (heavy pyrolysis oil (HPO) in FIG. 2) stream 150 can exit gas oil processing unit 130 and enter resid hydrocracking unit 144 to be further processed. Resid hydrocracking unit 144 can also produce pitch stream 152.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein can be utilized. Accordingly, the appended claims are intended to include within their scope such processes, machines, manufacture, compositions of matter, means, methods, or steps.

1. A system for the production of hydrocarbon products, the system comprising:
a crude oil processing unit capable of producing a gaseous hydrocarbon stream comprising hydrocarbons having a boiling point less than 200° C., a gas oil stream, and a heavy hydrocarbon stream;
a gaseous hydrocarbon separation unit capable of receiving the gaseous hydrocarbon stream comprising hydrocarbons having a boiling point less than 200° C. produced from the crude oil processing unit and capable of producing a naphtha stream, a gaseous stream comprising C4 hydrocarbons, and a C2-C3 hydrocarbon stream;
a steam cracking unit capable of receiving the C2-C3 hydrocarbon unit and cracking the C2-C3 hydrocarbon stream to produce ethylene and propylene;
a storage unit coupled to the gaseous hydrocarbon separation unit and capable of receiving the gaseous stream comprising C4 hydrocarbons; and
a storage unit coupled to the gaseous hydrocarbon separation unit and capable of receiving the naphtha.

2. The system of claim 1, wherein the crude oil processing unit comprises a crude distillation unit and a vacuum distillation unit.

3. The system of claim 1, further comprising:
a butene hydrogenation unit; and
a C4 conduit coupled to the steam cracking unit and the butane hydrogenation unit, the C4 conduit capable of providing C4 hydrocarbons from the butene hydrogenation unit to the steam cracking unit.

4. The system of claim 1, further comprising:
a resid hydrocracking unit capable of receiving the heavy hydrocarbon stream and configured to produce naphtha from the heavy hydrocarbon stream; and
a naphtha conduit coupled to the resid hydrocracking unit and the steam cracking unit, the naphtha conduit capable of providing naphtha from the resid hydrocracking unit to the steam cracking unit.

5. The system of claim 1, further comprising:
a hydrocracking unit capable of receiving the gas oil and configured to produce naphtha and unconverted oil (UCO) from the gas oil; and
a second naphtha conduit coupled to the hydrocracking unit and the steam cracking unit, the naphtha conduit capable of providing naphtha from the hydrocracking unit to the steam cracking unit.

6. A process for the production of hydrocarbons, the process comprising:
(a) subjecting a crude oil hydrocarbon feed to conditions sufficient to produce a gaseous hydrocarbon stream comprising hydrocarbons have a boiling point less than 200° C., a gas oil stream, and a heavy hydrocarbon stream;
(b) in a gaseous hydrocarbon unit, separating the gaseous hydrocarbon stream comprising hydrocarbons have a boiling point less than 200° C. into a C2-C3 hydrocarbon stream, a C4 hydrocarbon stream, and a naphtha stream;
(c) in a steam cracking unit, subjecting the C2-C3 hydrocarbons to conditions sufficient to produce an ethylene product stream and a propylene product stream; and
(d) storing the C4 hydrocarbon stream and the naphtha stream.

7. The process of claim 6, wherein the conditions in step (a) comprise a temperature of 50° C. to 700° C.

8. The process of claim 6, further comprising subjecting gas oil to conditions sufficient to produce a second naphtha stream and an unconverted oil (UCO) stream.

9. The process of claim 8, further comprising providing the second naphtha stream to the steam cracking unit of step (c).

10. The process of claim 9, further comprising providing the UCO stream to the steam cracking unit of step (c).

11. The process of claim 6, further comprising subjecting the heavy hydrocarbon to conditions sufficient to produce a third naphtha stream and pitch.

12. The process of claim 10, further comprising subjecting the second and third naphtha streams, the UCO stream or a combination thereof, to conditions in the steam cracking unit of step (c) sufficient to produce a second C4 hydrocarbon stream.

13. The process of claim 12, wherein the cracking conditions are different than the cracking conditions for the C2-C3 hydrocarbons.

14. The process of claim 12, wherein the second C4 hydrocarbon stream is further processed to produce methyl tert-butyl ether (MTBE).

15. The process of claim 14, further comprising providing one or more C4 hydrocarbons streams produced from a butene hydrogenation unit to the steam cracking unit of step (c).

16. The process of claim 15, further comprising subjecting the gaseous hydrocarbon stream of step (a) to conditions sufficient to lower a sulfur content of the gaseous hydrocarbon steam to less than 500 ppm.
